(19) United States
(12) Patent Application Publication
(10) Pub. No.: US 2005/0085762 A1
(43) Pub. Date: Apr. 21, 2005

(54) Perfusion circuit for cardiopulmonary bypass with air removal system

(76) Inventors: Venkataramana Vijay, Tarrytown, NY (US); Kenvin McCusker, Portsmouth, NH (US)

Correspondence Address: Gordon & Jacobson, P.C., 65 Woods End Road, Stamford, CT, 06905, US

(21) Appl. No.: 11/009,624

(22) Filed: Nov. 18, 2004

Related U.S. Application Data
(63) Continuation-in-part of application No. 10/403,567, filed on Mar. 31, 2003, now Pat. No. 6,852,280.

(51) Int. Cl. A61M 37/00; A61M 1/14; A61M 1/34; A61M 1/36

(52) U.S. Cl. 604/6.14; 422/45

(57) ABSTRACT

A condensed circuit for cardiopulmonary bypass includes an air removal system located after the junction of a shunt line and the reservoir, but before the perfusion pump. Both passive and active systems are described. The dual role of the air removal system is to remove/aspirate micro-air bubbles from the venous side of the circuit and to prevent large air boluses from entering the centrifugal pump and stopping the pump.

This application is a continuation-in-part of U.S. Ser. No. 10/403,567, filed Mar. 31, 2003 which is hereby incorporated by reference herein in its entirety.

### BACKGROUND OF THE INVENTION

1. Field of the Invention

This invention relates broadly to medical systems. More particularly, this invention relates to perfusion circuits for cardiopulmonary bypass and cardioplegia and methods of using the same.

2. State of the Art

In conventional open-heart surgery, the patient's breast bone is sawed open, the chest is spread apart with a retractor, and the heart is accessed through the large opening created in the patient's chest. The patient is placed on cardiopulmonary bypass and the patient's heart is then arrested using catheters and cannulae which are inserted directly into the large arteries and veins attached to the heart through the large opening in the chest.

Referring to prior art FIG. 1, the prior art bypass perfusion circuit 10 includes an arterial cannula 12 typically passed through the wall of the ascending aorta 14. A venous cannula 16 is passed through the right atrium 18 for withdrawing blood from the patient. The venous cannula 16 is coupled to an approximately eight foot length of ½ inch diameter polyvinyl chloride (PVC) tubing 20 (volume of 304 ml). All prior art systems have mandated the use of at least ½ inch diameter tubing at this location in order to ensure proper blood flow. Tubing 20 leads to a blood reservoir 22 adapted to store a blood volume of 300 to 600 ml. A one foot length of ⅜ inch diameter tubing 24 (volume of 21 ml) couples the reservoir 22 to a centrifugal pump 26 which has a volume of 80 ml. The centrifugal pump 26 is connected to a heart/lung console (not shown) which power the pump. A one foot length of ⅜ inch diameter tubing 28 (volume of 21 ml) transfers blood from the pump 26 to an oxygenator 30 (volume 280 ml). Another one foot length of ⅜ inch diameter tubing 32 (volume of 21 ml) transfers blood from the oxygenator 30 to a forty micron arterial filter 34 (volume of 50 ml). The arterial filter 34 is adapted to capture gaseous and fatty embolisms. From the filter 34, an eight foot length of ⅜ inch diameter tubing 36 (volume 168 ml) completes the circuit back to the arterial cannula 12. The reservoir 22, oxygenator 30, and arterial filter 34 are an integrated unit 38. Nevertheless, tubings 24, 28, 32 are required to connect the various sections thereof. Blood is pulled from the patient through the venous cannula 16, circulated through the tubing, reservoir, pump, oxygenator and filter, which are together referred to as the perfusion circuit 10, and back to the patient through the arterial cannula 12. The entire bypass circuit 10 is mounted on a pole fixed to the console (now shown) which powers the centrifugal pump 26, and is thus constrained to the location of the console.

Prior to use, the two lengths of eight foot tubing 20, 36 of the circuit 10 are coupled together at a pre-bypass filter (not shown) having an 80 ml volume. One length of the tubing is then decoupled from about the pre-bypass filter and the circuit is primed with an isotonic solution, e.g., saline, to remove air and any other impurities from within the components and tubing. The priming volume is relatively high, calculated from the above stated individual volumes of the tubing and components to be approximately 1325 to 1625 ml (not including the cannulae). Note that the ½ inch diameter tubing has a volume of 38 ml/foot, and ⅜ inch diameter tubing has a volume of 21 ml/foot.

After the perfusion circuit 10 is primed with saline, the pre-bypass filter is removed and respective ends of the circuit are coupled to the arterial and venous cannulae 12, 16.

Referring to prior art FIG. 2, a cardioplegia circuit 40 is then coupled to the heart. The cardioplegia circuit 40 generally includes a roller pump 42 which pulls blood from the oxygenator and feeds the blood into the heart. To the circuit 40, cardioplegia fluid 44 is added. The cardioplegia fluid 44 is generally potassium suspended in a one liter isotonic solution. A length of flexible tubing 46 extends from the roller pump 42 to a bubble trap 48 and a catheter 50 extending from the bubble trap 48 into the heart 15. The components and tubing of the cardioplegia circuit must also be primed with approximately 300 to 400 ml of an isotonic solution to remove air and foreign matter prior to use. After priming, the roller pump 42 is operated to induce cardioplegia.

Once the perfusion circuit pump is operated, cardioplegia is induced and the patient's blood is oxygenated outside the body and circulated back to the patient. When the perfusion pump is operated, the priming saline is also circulated through the patient's body.

This standard procedure is undesirable for several reasons. First, the relatively large priming volume, and particularly the length of tubing, of the system requires that the patient's blood come into contact with a large non-vascular surface area for a relatively long period of time. When blood contacts plasticizer components such as the tubing, there tends to be an inflammatory response by the body. This is so even when the tubing and other components are coated with modern anti-inflammatory coatings. This response can compromise the recovery of the patient.

Second, there are instances in prior art perfusion circuits where a section of the tubing or cannulae kinks, inhibiting blood flow through the perfusion circuit. In such a situation, the pump may draw in air through the reservoir, which is open to the atmosphere, and circulate the air into the patient's vascular system. This is extremely dangerous to the patient and may even be deadly.

Third, the large amount of isotonic fluid required for priming the perfusion bypass and cardioplegia circuits is circulated into the patient's body in addition to units of blood that may have been administered to the patient prior to the procedure. This extraordinary volume of fluid in the human body taxes the patient, as the kidneys are forced to process a substantial amount of additional fluid.

### SUMMARY OF THE INVENTION

It is therefore an object of the invention to provide a perfusion bypass circuit that minimizes priming volume.

It is another object of the invention to provide a perfusion circuit that prevents air from being pumped through the circuit in the event of cannulae or tubing kinks.

It is a further object of the invention to provide a combined perfusion and cardioplegia circuit that minimizes priming volume.

It is also an object of the invention to provide a method of using a perfusion circuit for cardiopulmonary bypass in which no priming liquid is circulated through the body.

It is an additional object of the invention to provide a method of using a perfusion circuit for cardiopulmonary bypass in which air is prevented from being circulated into the patient, even if a section of tubing or a cannulae is temporarily or permanently closed.

It is still another object of the invention to provide a method of introducing cardioplegia fluid which introduces relatively little, if any, additional isotonic fluid to the patient.

In accord with these objects, which will be discussed in detail below, a perfusion circuit for cardiopulmonary bypass and cardioplegia, and methods of using the same, are provided. The perfusion circuit includes a bypass portion including tubing and components which together have a substantially shorter path length and priming volume than prior art bypass perfusion circuits. The total priming volume of the bypass circuit in the preferred embodiment is under 800 ml, and more preferably under 700 ml. This reduction in priming volume is partially effected by reducing the volume of blood which will be pooled in the reservoir, as discussed below. As the length and volume of the circuit is relatively shorter and smaller than the prior art, there is reduction in the inflammatory response caused by blood contacting plasticizers.

According to another preferred aspect of the invention, a shunt is provided to connect the venous side one foot length of tubing with the pump. The shunt bypasses the reservoir. A releasable clamp is provided for the shunt. Releasable clamps are provided before and after the reservoir to prevent blood flow thereto or therefrom when the shunt is opened.

According to another preferred, but optional, aspect of the invention, a condensed cardioplegia circuit is provided. The cardioplegia circuit includes a roller pump having an input coupled to an output port of the oxygenator, and an output coupled to a bubble trap by a very short piece of tubing. A catheter is coupled to the bubble trap and includes a needle at its end which is inserted into the heart. In accord with the invention, an infusion pump is provided which infuses concentrated cardioplegia fluid into the circuit, without necessitating large volumes of isotonic solution which would otherwise be pumped into the patient. Thus, the circuit described is a total circuit for both bypass and cardioplegia.

In accord with a preferred method of the invention, the circuit is primed with a saline and operated to circulate the saline through the pre-bypass filter. After circulation of the saline, the venous-side and arterial-side tubings are separated from the pre-bypass filter, and coupled to respective venous and arterial cannulae which are inserted into the heart.

The saline is then replaced with the patient's own blood. That is, blood is pulled from the venous and arterial sides into the bypass circuit and the saline is drained by disconnecting tubing from a component or via a valve. This is permissible due to the small priming volume of the bypass circuit. In prior art systems, a sufficiently large volume of priming solution is required such that it would not be medically safe to prime the circuit with the patient's blood. Where the patient's blood can be the priming fluid, none of the negative side effects associated with circulating a large volume of non-blood fluid through the body results. Preferably, 0 to 100 ml of blood is pooled in the reservoir.

Once the bypass circuit is primed with blood, the bypass circuit is activated. The bypass circuit circulates the patient's blood from the reservoir through the oxygenator and the arterial filter, and then back to the patient.

Then, the cardioplegia roller pump pulls blood from the oxygenator, and sends it through the bubble trap. The blood in the cardioplegia circuit is infused with cardioplegic fluid from the infusion pump and circulated to the heart. Practically no saline solution is required for the cardioplegia portion of the total circuit. In addition, all saline solution can be eliminated from entering the patient by also replacing the saline solution in the cardioplegia circuit with blood prior to operating the infusion pump.

According to one preferred aspect of the invention, where a patient has sufficient blood, such as when additional blood units have been administered prior to the procedure, the reservoir is permitted to pool a reserve of blood. Then the shunt clamp is opened and the reservoir clamps are closed to remove the blood in the reservoir from the circulation. The perfusion pump then pulls blood directly from the patient and through the shunt, while bypassing the reservoir. Then, if any of the tubing on the arterial side of the pump kinks, the venous cannula will apply suction against the heart tissue and close circulation. No air can enter from the reservoir. Moreover, if any of the tubing on the venous side of the pump kinks, the physician or perfusionist can release the reservoir clamps and again allow the pump to pull blood from the reservoir until the kinking can be resolved. If any blood remains in the reservoir at the end of the bypass procedure, the reservoir clamp can be released and the arterial side of the circuit may be clamped close to cause substantially all of the reservoir blood to be reintroduced into the patient.

From the foregoing, it is appreciated that a condensed and safe circuit is provided for bypass and cardioplegia.

Additional objects and advantages of the invention will become apparent to those skilled in the art upon reference to the detailed description taken in conjunction with the provided figures.

### BRIEF DESCRIPTION OF THE DRAWINGS

Prior art FIG. 1 illustrates a prior art bypass perfusion circuit;

Prior art FIG. 2 illustrates a prior art cardioplegia perfusion circuit;

FIG. 3 illustrates a total condensed circuit according to the invention which provides bypass and cardioplegia perfusion;

FIG. 4 is a schematic illustration of the total condensed circuit of FIG. 3 mounted on a pole which is coupled to a heart/lung console with an articulating arm.

FIG. 5 is a broken illustration of a total condensed circuit according to the invention provided with a first embodiment of an air removal system;

FIG. 6 is a schematic illustration of a second embodiment of an air removal system according to the invention;

FIG. 7 is a schematic illustration of a third embodiment of an air removal system according to the invention;

FIG. 8 is a schematic illustration of a fourth embodiment of an air removal system according to the invention;

FIG. 9 is a schematic illustration of a fifth embodiment of an air removal system according to the invention; and

FIG. 10 is a schematic illustration of a sixth embodiment of an air removal system according to the invention.

### DETAILED DESCRIPTION OF THE PREFERRED EMBODIMENTS

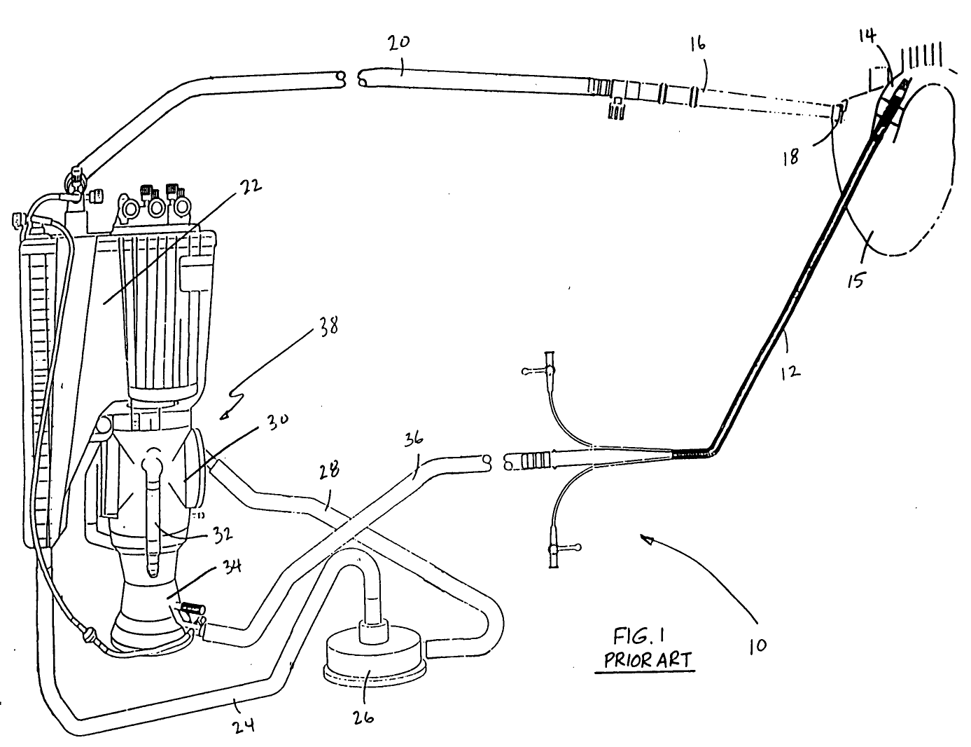

Turning now to FIG. 3, a low priming volume combined heart-bypass and cardioplegia circuit, hereinafter referred to as a total condensed circuit 100, is shown. The circuit 100 includes bypass portion 102 and a cardioplegia portion 104, which will be described in detail below.

The bypass portion 102 includes a venous-side approximately one foot long ⅜ inch diameter PVC tubing 106 (volume of 21 ml) which leads to a first Y-connector 108. From the Y-connector 108, an approximately three inch length of ⅜ diameter tubing 110 (volume 5 ml) is coupled to a blood reservoir 112 (which as discussed below will preferably hold 0 to 100 ml of blood). The blood reservoir 112 is preferably open to (or openable to) atmosphere. Tubing 110 is provided with a releasable clamp 114. Another short three inch length of ⅜ inch diameter tubing 116 (volume 5 ml), provided with a releasable clamp 118, extends from the reservoir 112 and is coupled to a second Y-connector 120. Another short three inch length of ⅜ inch diameter tubing 122 (volume 5 ml) extends from the second Y-connector 120 to a first perfusion pump 124 (volume 80 ml), thereby placing the reservoir 112 and the pump 124 in fluid communication. A six inch length of ⅜ inch diameter tubing 126 (volume 10 ml) transfers blood from the pump 124 to an oxygenator 128 (volume 280 ml). Another six inch length of ⅜ inch diameter tubing 130 (volume 10 ml) transfers blood from the oxygenator 128 to an arterial filter 132 (volume 50 ml). In order to use such a short length of tubing between the pump 124 and the oxygenator 128, the location of the pump 124 is relocated adjacent the arterial filter 132. An arterial-side one foot length of ⅜ inch diameter tubing 134 (volume 21 ml) extends from the arterial filter 132. Prior to priming, the venous- and arterial-side tubing 106, 134 are coupled at a pre-bypass filter (not shown, volume 80 ml). Therefore, the total priming volume of the bypass circuit 102 in a preferred embodiment is under 800 ml, more preferably under 700 ml, and most preferably between approximately 567 to 670 ml. This reduction in priming volume is effected by reducing the diameter of the venous side tubing (contrary to prior art teachings), reducing the length of the tubing, relocating the components relative to the each other, and reducing the volume of blood which will be pooled in the reservoir, as discussed with respect to the method described below. As the length and volume of the bypass circuit is relatively shorter and smaller than the prior art, there is less opportunity for an inflammatory response caused by blood contacting plasticizers.

The bypass circuit 102 is preferably, though optionally, provided with a shunt 136. The shunt 136 is preferably a twelve to eighteen inch length of ⅜ inch diameter tubing (volume 20 to 30 ml) connecting the first Y-connector 108 with the second Y-connector 120 and thereby bypassing the reservoir 112. A releasable clamp 138 is provided on the shunt 136. The use of the shunt 136 is described below with respect to the method of the invention.

The cardioplegia circuit 104 includes a relatively short length of 3/16 to ¼ inch diameter tubing 154 coupled to an output port 152 of the oxygenator 128. Tubing 154 or a tubing 158 coupled thereto extends through a roller pump 150 which is adapted to move rollers against the tubing to thereby pull blood into the tubing and move it therethrough. After the tubing 158 exits the roller pump 150, it is coupled to a bubble trap 156. A catheter 160 is coupled to the bubble trap 156, and a needle 164 is provided at the end of the catheter 160 and is adapted to be inserted into the heart 166. The circuit 104 also includes an infusion pump 168 which slowly infuses a small volume of highly concentrated cardioplegic fluid (e.g., potassium in solution) into the tubing 158. Preferably, only 5 to 40 ml of the concentrated cardioplegia fluid is introduced into the patient over the course of the bypass procedure, as opposed to 1 to 2 liters of cardioplegic solution in the prior art. As the tubing 154, 158 and catheter 160 are adapted to carry the patient's own blood from the oxygenator 128, the infusion pump 168 is adapted to infuse the concentrated cardioplegic fluid into the blood. This cardioplegia circuit has minimal priming volume, preferably no more than 30 to 40 ml.

The bypass circuit 102 is vertically mounted on a pole 170 with bar 172. The cardioplegia circuit 104 is mounted on the pole 170 at bars 174 and 176. The pole is self-supporting on wheels 184. Other mounting means may certainly be used. However, it is desirable that any mounting means permit the bypass circuit 102 and the cardioplegia circuit 104 to be located as close to the patient as possible to minimize the length of the tubing of the circuits.

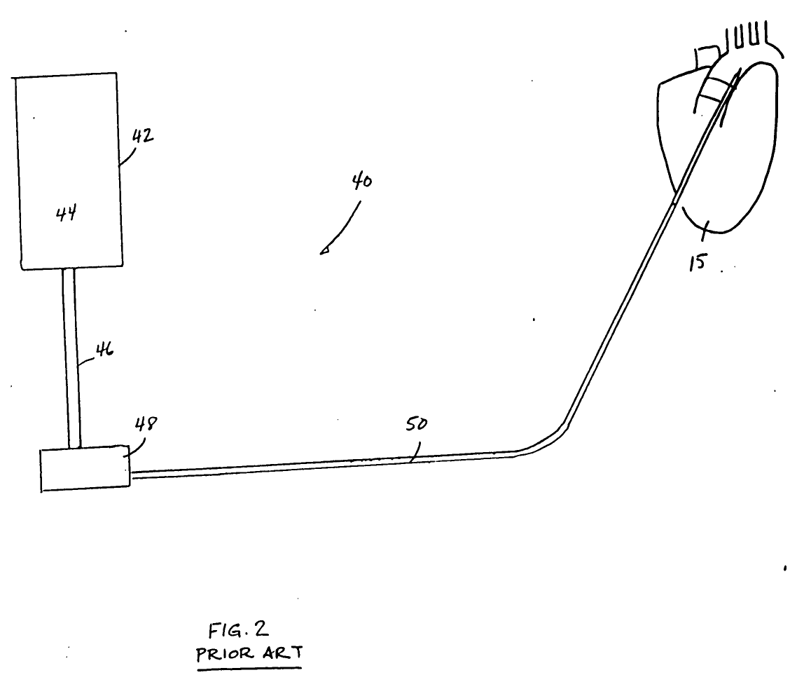

Referring to FIG. 4, according to one preferred embodiment of the invention, the pole 170 is coupled to the heart/lung console 180 by an articulating arm 182. The console 180, among other functions, powers the centrifugal pump 124 and provides controls therefor. The arm 182 maintains a structural relationship between the circuits 102, 104 and the console 180, while permitting a wide range of movement of the circuits relative to the console. Thus, the relatively large footprint console 180 can be located away from the patient to permit physician access to the patient, and the relatively small footprint of the pole 170 can be moved on its wheels 184 adjacent the patient, thereby permitting the very short tubing lengths of tubes 106 and 134. The arm 182 ensures that the pole 170 is not moved too far relative to the console 180, i.e., the pole is not moved a distance which may cause inadvertent disconnect between the centrifugal pump 124 and the console 180. Furthermore, the arm 182 provides a psychological benefit in that the circuits 102, 104 and console 180 maintain the appearance of an integrated unit. Alternatively, a preferably cantilevered swing-arm (not shown) mounted to the console may be provided, and the circuits 102, 104 can be mounted to a pole which is supported by the swing-arm. This system also permits the circuits to be located at a distance relative to the console and, more importantly, closer to the patient.

Referring back to FIG. 3, in accord with a preferred method of the invention, the bypass circuit is primed with an isotonic priming solution, e.g. saline, which is circulated by the pump 124 through the pre-bypass filter. After priming and circulation, the venous-side and arterial-side tubings 106, 134 are separated from the pre-bypass filter (not shown). The pre-bypass filter is then removed and venous and arterial cannulae 180, 182 are coupled to the respective venous- and arterial-side tubing 106, 134 and inserted into the heart 166.

In accord with a preferred aspect of the invention, the isotonic solution is then replaced with the patient's own blood. That is, using gravity or vacuum-assist, blood is pulled into the venous and arterial sides of the bypass circuit 102 and the saline is released by disconnecting tubing from a component (e.g., at tubing 122 and at tubing 134) or via a valve. This is permissible due to the small priming volume of the bypass circuit 102. In prior art systems, a sufficiently large volume of priming solution is required such that it would not be medically safe to prime the circuit with the patient's blood. However, in the present invention, since the patient's blood can be the priming fluid, none of the negative side effects associated with circulating a large volume of non-blood fluid through the body results. Preferably, in addition to priming the circuit, 0 to 100 ml of blood is pooled in the reservoir 112. This is also in contradiction to prior art methodology, where it is standard to pool at least 300 ml, and up to 600 ml, of blood during operation of the bypass circuit.

Once the bypass circuit 102 is primed with blood, the bypass circuit is activated. The pump 124 in the bypass circuit 102 circulates the patient's blood from the reservoir 112 to the oxygenator 128, the arterial filter 132, and then back to the patient through the arterial-side tubing 134 and cannula 182.

After bypass is activated, cardioplegia is induced. This is effected via the condensed cardioplegia circuit 104, in which the perfusion roller pump 150 pulls blood from the oxygenator 128, and sends it through the bubble trap 156 and then to the heart of the patient. The blood in the circuit, e.g., either before the roller pump 150 or exiting the bubble trap 156, is infused with cardioplegic fluid (concentrated potassium in solution) from the infusion pump 168 and circulated to the heart 166. Practically no isotonic priming solution is required for the cardioplegic portion of the total circuit 100. In addition, all priming solution can be eliminated from the patient by replacing the priming solution in the cardioplegic circuit 104 with blood prior to operating the perfusion pump 150 and infusion pump 168. In this manner, the patient's own blood is the primary carrier for the cardioplegia fluid.

According to one preferred aspect of the invention, where a patient has sufficient blood, such as when additional blood units have been administered prior to the procedure, the reservoir 112 is permitted to pool a reserve of blood, e.g., up to approximately 3000 ml, although the amount of stored blood volume is dependent upon the flow and oxygenation needs of the patient during the procedure. Then the shunt clamp 138 is opened and the reservoir clamps 114, 118 are closed to remove the blood in the reservoir 112 from circulation. The bypass perfusion pump 124 then pulls blood directly from the patient and through the shunt 136, while bypassing the reservoir 112. Then, if any of the tubing on the arterial side of the pump 124 or the arterial cannula 182 kinks, the venous cannula 180 will apply suction against the heart tissue 166 and close circulation. No air can enter from the reservoir 112; thus, no air can be circulated into the patient. Moreover, if any of the tubing 106 on the venous side of the pump 124 kinks such that blood flow through the shunt is limited or prevented, the physician or perfusionist can release the reservoir clamps 114 and 118 and again allow the pump 124 to pull blood from the reservoir 112 until the kinking can be resolved.

If any blood remains in the reservoir at the end of the bypass procedure, the reservoir clamp can be released and the arterial side of the circuit may be clamped close to cause substantially all of the reservoir blood to be reintroduced into the patient.

According to another aspect of the invention, an air removal system is shown which facilitates removing air from the circuit when the blood reservoir is excluded. It is appreciated that when the blood reservoir is within the circuit, the blood reservoir operates to remove any air entrained in the blood. However, when the blood reservoir is excluded from the circuit, it is important to ensure that all air is removed from the circuit. If air enters the system, such air can trigger a sensor which stalls the centrifugal pump, shutting down the entire bypass system.

Limiting air entry into the circuit involves addressing all points of potential air entry into the circuit on the venous side, e.g., from the reservoir and from the cannular insertion into the heart. Even addressing such sites it is still possible that air may enter the system, e.g., when a surgeon repositions the cannula during surgery.

In accord with the invention, an air removal site is located distal to the junction of the shunt line and the reservoir, but proximal to the centrifugal pump. The dual role of the air removal system is to remove/aspirate micro-air bubbles from the venous side of the circuit and to prevent large air boluses from entering the centrifugal pump and stopping the pump. The air removal system may be used in any bypass circuit with shunt, regardless of priming volume, and is particularly useful in circuits having priming volumes less than 1000 ml, and more particularly useful in circuits of the preferred volume of 800 ml or smaller. Most preferably (though not necessarily), as described in the following embodiments, the air removal system provides passive separation of air from blood.

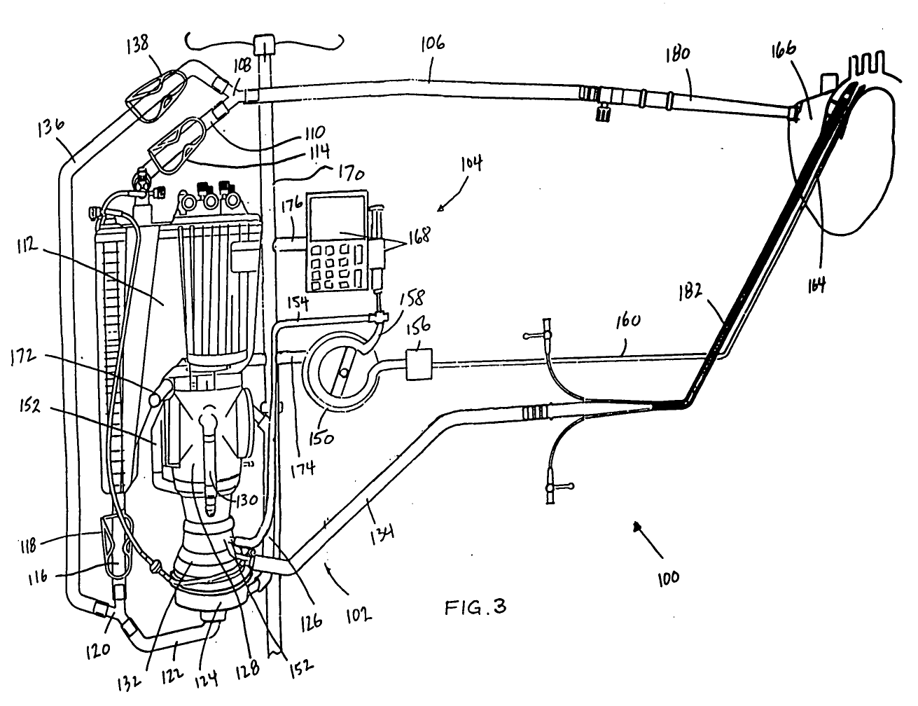

Turning now to FIG. 5, a portion of a condensed bypass system 200 according to the invention is shown, generally as described above with respect to system 100. An air removal system 228 is provided after the Y-connector 220 joining the shunt line 236 and the tubing 216 at the drain of the reservoir 212, and before the centrifugal pump 224. More particularly, the air removal system 228 includes an air trap 230 coupled between the connector 220 and the pump 224 with flexible tubing 232, 234. The air trap 230 includes an elongate main stem 237 and a side branch 238. The main stem 237 preferably includes graduated markings 240 and is evacuated via a preferably flexible ¼ or ⅜ inch diameter tubing 242 to an inlet 244 at the top of the blood reservoir 212. The side branch 238 is preferably provided with a luer connector 246 which receives a syringe 248 adapted to aspirate blood and air. The lower end of the air trap 230 preferably includes a T-connector 249 for joining tubing 232, 234. A first clamp 250 is provided for compressing tubing 234 to inhibit the flow of blood therethrough. A second clamp 252 is normally in a closed position about the lower end of the tubing 242 adjacent the air trap 230. The second clamp is preferably closed on a portion of tubing 242 which is filled with blood.

Air removal system 228 is a passive system, where when air is identified before the pump 224 such air may be forced up the air trap 230 by increasing impedance to flow through tubing 234 with the clamp 250 and releasing clamp 252. This causes blood and air to back up into the collector 230. By allowing a column of blood to rise into the collector 230 and the tubing 242, collected air is displaced into the reservoir 212. Alternatively, air may be manually aspirated from the collector 230 at side branch 238 with the syringe 248.

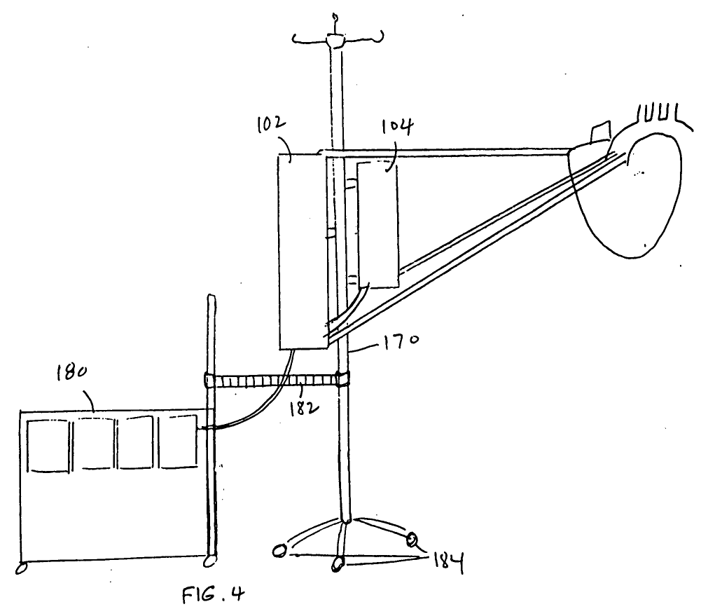

Referring to FIG. 6, another air removal system 328 is shown. Air removal system 328 includes substantially the same components as system 228, even if not shown. In general, an air trap 330 with main stem 337 and side branch 338 is situated after the shunt line 336 and reservoir 312 and before the centrifugal pump 324. System 328 further includes a preferably pediatric size roller pump 354 or other appropriate occluding siphon pump about evacuation tubing 342 which actively siphons blood and air up the air trap 330 and back into the reservoir 312. A clamp 352 is provided about a blood-filled portion of the evacuation tubing 342 between siphon pump 354 and the air trap 330. When air rises into the air trap 330, clamp 350 is activated to inhibit flow therethrough, clamp 352 is released to permit flow up the evacuation tube 342, and the siphon pump 354 is activated to cause blood and air to be siphoned through the air removal system 328 and back into the reservoir.

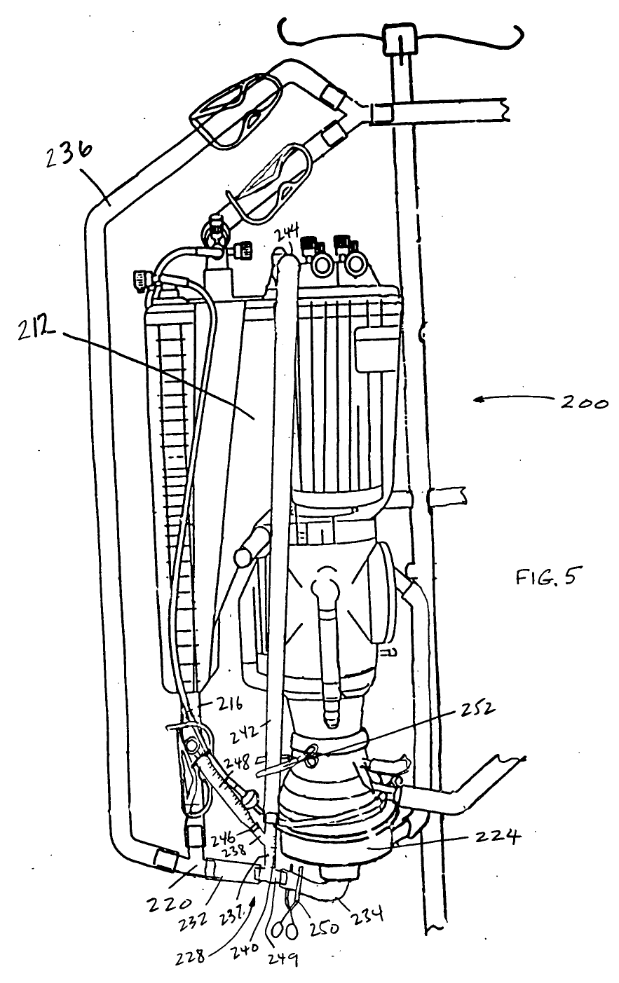

Turning now to FIG. 7, another air removal system 428 is shown. Air removal system 428 includes a collector 430 having a main stem 437 with an upper branch 438 having a luer connector for coupling with a syringe 448 and a lower inverted Y-portion 456 for coupling shunt line feed 432 with tubing 434 through branches 458, 460 of the Y-portion 456. The Y-portion 456 also includes two branches 462, 464. The passages in the collector 430 are all preferably ¼ inch in diameter. A preferably ⅜ inch diameter U-shaped loop 466 of flexible tubing is coupled between the branches 462, 464 of the Y-portion of the collector 430 forming right and left air trap horns 468, 470. In normal operation, clamp 456 is open permitting uninhibited flow through tubing 466.

During normal operation of the bypass circuit, the left and right horns 468, 470 provide a means for air to rise to the top of the U-shaped loop 466 and up the air trap 430 which is clamped off by clamp 452. If sufficient air rises up the collector such that it needs to be evacuated, clamp 456 can be closed, clamp 452 can be opened, and clamp 450 can be tightened to inhibit but not prevent flow through tubing 434 so as to cause blood flow up the air trap and up the evacuation tube. Then, siphon pump 454 may be activated to facilitate siphoning blood into the blood reservoir 412. Once the air is removed from the evacuation tube 442, the siphon pump 454 is deactivated, clamp 452 is closed, and clamps 450 and 456 are released to permit blood to flow normally through the circuit; i.e., from tubing 432 through branch 462 of the air trap 430 through U-loop 466 through branch 464 of the air trap through tubing 434 to the pump 424.

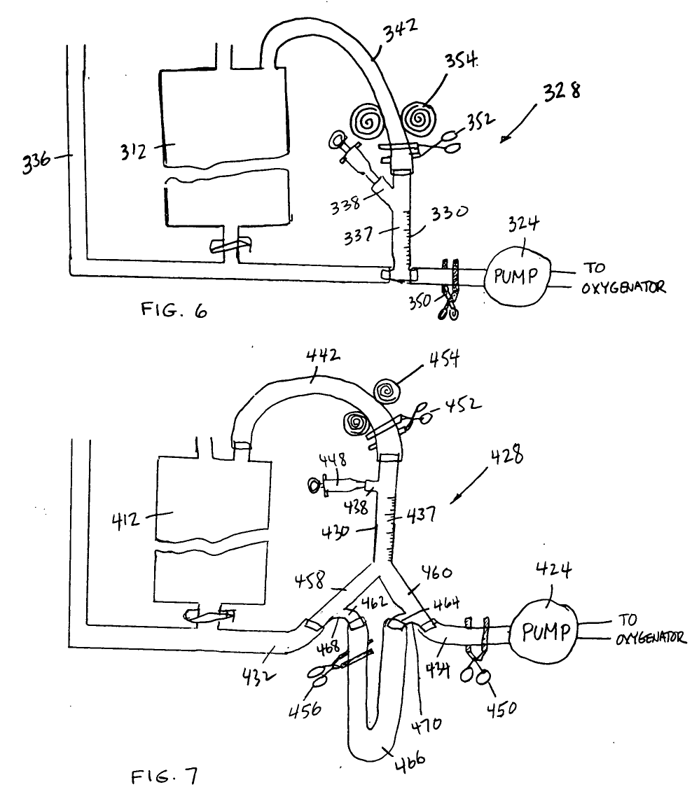

Referring now to FIG. 8, another embodiment of an air removal system 528 is shown. The system 528 includes a canister 576 coupled between tubing 532 and 534. The canister 576 is substantially larger in diameter than tubings 532 and 534. By way of example, tubings 532, 534 are preferably approximately ⅜ inch in diameter, and canister is preferably approximately greater than one inch in diameter. While tubing 532 is shown coupled centrally at the side of canister 576 (rather than at an end) and entering transversely (it is preferably though not necessarily oriented at a perpendicular or oblique angle relative to the longitudinal axis of the canister 576), it may alternatively be coupled at an upper location at 577a or lower location 577b, or even at an end given the preferred substantial change in diameter. The canister 576 operates to introduce turbulence before outputting the blood into tubing 534 toward the pump 524, preferably by changing the direction of blood flow. The introduced turbulence causes any air in the blood to collect at the top of the canister, e.g., at dome portion 578. A filter 580 may be provided within the canister 576 as an additional bubble/debris trap. Furthermore, when an impedance to blood flow is applied at tubing 534 at the inflow of the pump 524 (for example with clamp 550 to restrict or momentarily stop flow), the suction force developed by the pump 524 is limited and blood drains from the shunt 536 by the lesser force of gravity rather than by the substantially greater negative pressure of the pump 524. As there is no significant pushing/pulling force on the blood or air bubbles within, the blood may become momentarily relatively still within the canister 576 allowing the air bubbles to separate easily from the blood. The air trap 530, other clamps, and optionally siphon pumps and syringes discussed above in prior embodiments are also preferably arranged and used in the present embodiment.

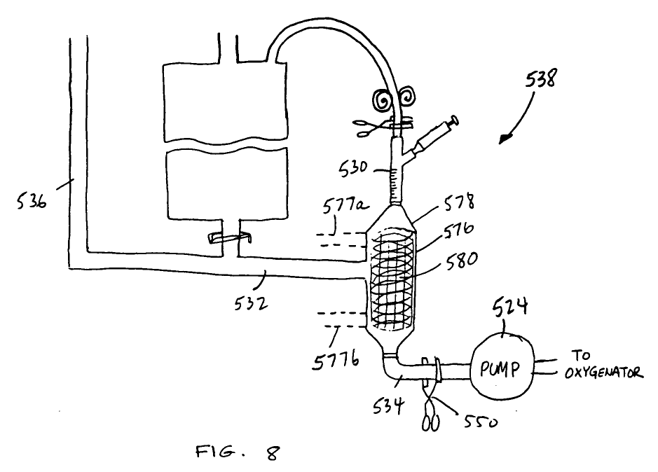

Turning now to FIG. 9, an air removal system 628 similar to the air removal system 428 shown in FIG. 7 is provided. Air removal system includes U-tubing 666 with bulb portions 682, 684 (preferably approximately 3 cm in diameter) which function like canister 476, and an acute angle change 685 in the blood flow path. These operate to induce some turbulent blood flow therein, and change the direction of blood flow. When the direction of blood flow changes and/or an impedance to blood flow is applied, air is separated out from the blood and rises upward into branches 686, 688, respectively. The branches 686, 688 are coupled to Y-connector 630 from which air can be siphoned back into the reservoir 612. A filter 690 may provided in bulb portion 684 (as shown) and/or bulb portion 682.

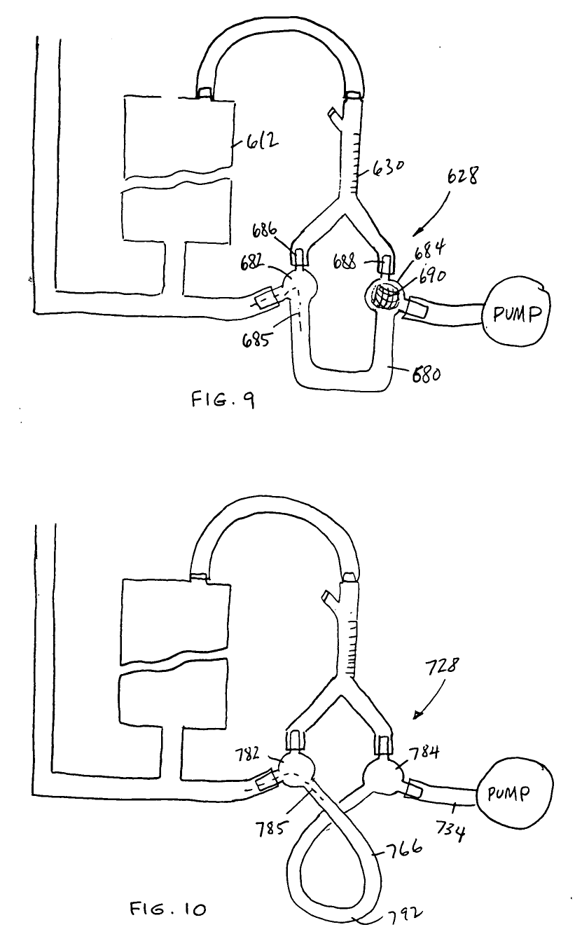

FIG. 10 shows an air removal system 728 substantially similar to system 628, but the tubing 766 is modified relative to tubing 666 to include a twist 792. Such twist permits a smaller change in the angle 785 of the blood flow path thereby reducing the turbulence relative to angle 685 (FIG. 9). When an impedance to blood flow is applied to the tubing 734, the bulbs 782, 784 collect the air. The smoother path 786 and reduced turbulence prevent cellular damage to the blood and also prevent blood clotting along the lumen of the tubing 766.

From the foregoing, it is appreciated that a condensed and safe circuit is provided for bypass and cardioplegia.

There have been described and illustrated herein embodiments of a total condensed circuit, i.e., a combined bypass and cardioplegia circuit of substantially small priming volume, and methods of heart-lung bypass and inducing cardioplegia. While particular embodiments of the invention have been described, it is not intended that the invention be limited thereto, as it is intended that the invention be as broad in scope as the art will allow and that the specification be read likewise. Thus, while a total condensed circuit has been disclosed, it will be appreciated that the bypass circuit may be constructed and used without the cardioplegia circuit. Moreover, while a circuit having the low priming volumes described is preferred where the patient's blood is used to prime the circuit, it should be understood that any method of bypass and/or cardioplegia where the patient's blood is used to prime the circuit, regardless of the volume of the circuit, is within the scope of the invention. In addition, while one foot long sections are tubing are provided for coupling the venous and arterial sides of the circuit to the respective cannulae, it is appreciated that longer tubing, e.g., up to approximately two feet, may be used to facilitate placement of the circuit relative to the patient. Even by extending this tubing by such length, only an additional 40 ml of priming volume is added to the circuit. It will therefore be appreciated by those skilled in the art that yet other modifications could be made to the provided invention without deviating from its spirit and scope as claimed.

1. A cardiopulmonary bypass circuit for oxygenating blood outside a patient and circulating oxygenated blood through the patient, said circuit comprising:
a) a venous line;
b) a blood reservoir in fluid communication and in series with said venous line;
c) a perfusion pump in fluid communication and in series with said blood reservoir;
d) a blood oxygenator in fluid communication and in series with said pump;
e) a shunt placing said venous line and said pump in fluid communication and bypassing said blood reservoir; and
f) structure external said reservoir which prevents any store of blood in said blood reservoir from being pulled by said perfusion pump when said blood reservoir is excluded from said circuit and said perfusion pump draws blood from the patient through said shunt such that said perfusion pump pulls blood from said venous line while bypassing the any store of blood in said blood reservoir.
g) an air removal means provided between said shunt and said perfusion pump which removes air bubbles from blood circulating in said circuit.

2. A cardiopulmonary bypass circuit according to claim 1, wherein:
said air removal means includes a pathway having a vertical air trap in fluid communication with said circuit, and which evacuates into said blood reservoir.

3. A cardiopulmonary bypass circuit according to claim 2, wherein:
said pathway includes a connector means for coupling a syringe to said pathway to aspirate air from said pathway.

4. A cardiopulmonary bypass circuit according to claim 2, wherein:
said lower vertical portion is provided with graduated markings.

5. A cardiopulmonary bypass circuit according to claim 2, wherein:
said air removal means includes a flexible tubing extending from said lower vertical portion to said blood reservoir.

6. A cardiopulmonary bypass circuit according to claim 5, wherein:
said lower vertical portion and said flexible tubing are of different diameters.

7. A cardiopulmonary bypass circuit according to claim 5, further comprising:
a releasable clamp provided between said perfusion pump and said air removal means.

8. A cardiopulmonary bypass circuit according to claim 5, further comprising:
a siphon pump provided about said tubing.

9. A cardiopulmonary bypass circuit according to claim 8, further comprising:
a releasable clamp provided about said tubing between said siphon pump and said lower vertical portion.

10. A cardiopulmonary bypass circuit according to claim 1, wherein:
said air removal means include a luer connector.

11. A cardiopulmonary bypass circuit according to claim 1, further comprising:
said air removal means includes an air trap having an upper end and first and second lower branches, said upper end coupled to an air evacuation tube which removes air from said circuit, said first lower end coupled to said shunt and said second lower end coupled to said perfusion pump.

12. A cardiopulmonary bypass circuit according to claim 11, wherein:
said air evacuation tube is coupled to said blood reservoir.

13. A cardiopulmonary bypass circuit according to claim 11, wherein:
said air trap includes graduated markings.

14. A cardiopulmonary bypass circuit according to claim 11, further comprising:
a loop of tubing also coupled between said first and second branches of said air trap.

15. A cardiopulmonary bypass circuit according to claim 14, wherein:
said loop of tubing is U-shaped.

16. A cardiopulmonary bypass circuit according to claim 15, wherein:
said loop of tubing and said first and second branches forming at least portions of first and second air trap horns.

17. A cardiopulmonary bypass circuit according to claim 11, wherein:
said air trap is provided with a connector means for coupling a syringe to said air trap.

18. A cardiopulmonary bypass circuit according to claim 11, further comprising:
a siphon pump coupled to said evacuation tube.

19. A cardiopulmonary bypass circuit according to claim 18, further comprising:
a releasable clamp provided about said evacuation tube between said siphon pump and said air trap.

20. A cardiopulmonary bypass circuit according to claim 11, further comprising:
a releasable clamp provided between said perfusion pump and said air trap.

21. A cardiopulmonary bypass circuit according to claim 11, further comprising:
tubing coupled between said first and second branches of said air trap, and
means to induce turbulent blood flow within a portion of said tubing.

22. A cardiopulmonary bypass circuit according to claim 21, wherein:
said means to induce turbulent blood flow includes at least one bulb portion having a relatively larger diameter than an adjacent portion of said tubing.

23. A cardiopulmonary bypass circuit according to claim 21, further comprising:
a filter provided in said means to induce turbulent blood flow.

24. A cardiopulmonary bypass circuit according to claim 1, further comprising:
means to induce turbulent blood flow in a portion of said air removal system.

25. A cardiopulmonary bypass circuit according to claim 24, wherein:
said air removal system includes an input to and output from said means to induce turbulent blood flow, and said diameter of said input is smaller than said diameter of said means to induce turbulent blood flow.

26. A cardiopulmonary bypass circuit according to claim 24, wherein:
said means to induce turbulent blood flow extends in a first direction, and an output from said shunt enter said means to induce turbulent blood flow in an orientation transverse to said first direction.

27. A cardiopulmonary bypass circuit according to claim 24, further comprising:
a filter provided in said means to induce turbulent blood flow.

28. A cardiopulmonary bypass circuit according to claim 1, wherein:
said air removal means changes a direction of flow of blood within said bypass circuit.

29. A cardiopulmonary bypass circuit according to claim 1, wherein:
said air removal means is a passive system.

30. A cardiopulmonary bypass circuit according to claim 1, further comprising:
structure external said reservoir which prevents any store of blood in said blood reservoir from being pulled by said perfusion pump when said blood reservoir is excluded from said circuit and said perfusion pump draws blood from the patient through said shunt such that said perfusion pump pulls blood from said first length of tubing while bypassing the any store of blood in said blood reservoir.

31. A cardiopulmonary bypass circuit according to claim 1, wherein:
said circuit has a priming volume of less than approximately 1000 ml.

32. A method of removing air from a cardiopulmonary bypass circuit for oxygenating blood outside a patient and circulating oxygenated blood through the patient, said circuit having a venous line, a blood reservoir in fluid communication and in series with said venous line, a perfusion pump in fluid communication and in series with said blood reservoir, a blood oxygenator in fluid communication and in series with said pump, a shunt placing said venous line and said pump in fluid communication and bypassing said blood reservoir, said method comprising:
restricting the flow of the circulating blood by creating impedance to blood flow into the pump, said restricting the flow permitting air to separate from the blood; and
removing the separated air from the circuit.

33. A method according to claim 32, wherein:
said restricting the flow occurs after the shunt.

34. A method according to claim 32, wherein:
said restricting the flow includes momentarily stopping said flow.
